(19) United States
(12) Patent Application Publication
(10) Pub. No.: US 2014/0199749 A1
(43) Pub. Date: Jul. 17, 2014

(54) Compositions and Methods for cDNA Synthesis

(71) Applicant: Jun Euihum Lee, San Diego, CA (US)

(72) Inventor: Jun Euihum Lee, San Diego, CA (US)

(21) Appl. No.: 13/871,810

(22) Filed: Apr. 26, 2013

Related U.S. Application Data
(60) Provisional application No. 61/752,617, filed on Jan. 15, 2013.

(51) Int. Cl. C12N 9/12; C12N 9/96

(52) U.S. Cl. CPC C12N 9/1276; C12N 9/96
USPC 435/188

(57) ABSTRACT

The present invention relates to methods and compositions for preparing cDNAs, and more particularly, compositions having trehalose for synthesizing a cDNA molecule or molecules from an mRNA template or population of mRNA templates under conditions sufficient to increase the detection sensitivity and cDNA yield and to simplify and improve the reliability of reverse transcription. The reagent mixture comprises a ready to use reagent solution, wherein the solution comprises: (a) trehalose in a concentration between about 5% and about 35%; and (b) a viral reverse transcriptase selected from the group consisting of AMV RT, RSV RT, MMLV RT, HIV RT, EIAV RT, RAV2 RT, ASLV RT, RNaseH (−) RT, SuperScript II RT, and ThermoScript RT, in a buffer suitable for use in a reverse transcription reaction, wherein the buffer further comprises a co-factor metal ion and nucleoside triphosphates.

### CROSS REFERENCE TO RELATED APPLICATIONS

This application claims priority to Provisional Application Ser. No. 61/752,617, filed Jan. 15, 2013, the contents of which are incorporated herein by reference in their entirety.

### FIELD OF THE INVENTION

The present invention relates to methods and compositions for preparing cDNAs, and more particularly, compositions having trehalose for synthesizing a cDNA molecule or molecules from an mRNA template or population of mRNA templates under conditions sufficient to increase the detection sensitivity and cDNA yield and to simplify and improve the reliability of reverse transcription.

### BACKGROUND OF THE INVENTION

The genetic framework of an organism is encoded in the double-stranded sequence of nucleotide bases in the deoxyribonucleic acid (DNA) and the genetic content of a particular segment of DNA, or gene, is manifested only upon production of the protein encoded by the gene. To produce a protein, one strand of the DNA is copied to produce a specific sequence of ribonucleic acid (RNA) and this particular type of RNA is called messenger RNA (mRNA).

Within a given cell, tissue or organism, there exist many mRNA species, each encoding a separate and specific protein, and the identity and levels of specific mRNAs present in a particular sample provides clues to the biology of the particular tissue or sample being studied. Therefore, the detection, analysis, transcription, and amplification of RNAs are among the most important procedures in modern molecular biology.

A common approach to the study of gene expression is the production of complementary DNA (cDNA). In this technique, the mRNA molecules from an organism are isolated from an extract of the cells or tissues of the organism. From these purified mRNA molecules, cDNA copies may be made using the enzyme reverse transcriptase (RT), which results in the production of single-stranded cDNA molecules. The term “reverse transcriptase” describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA which can then be cloned into a vector for further manipulation.

Avian myoblastosis virus (AMV) reverse transcriptase was the first widely used RNA dependent DNA polymerase (Verma, Biochem. Biophys. Acta 473:1 (1977)). The enzyme has 5′-3′ RNA directed DNA polymerase activity, 5′-3′ DNA directed DNA polymerase activity, and RNase H activity. RNase H is a processive 5′ and 3′ ribonuclease specific for the RNA strand for RNA DNA hybrids (Perbal, A Practical Guide to Molecular Cloning, New York: Wiley & Sons (1984)). A detailed study of the activity of AMV reverse transcriptase and its associated RNase H activity has been presented by Berger, et al., Biochemistry 22:2365-72 (1983). Another reverse transcriptase which is used extensively in molecular biology is reverse transcriptase originating from Moloney murine leukemia virus (M-MLV). See, e.g., Gerard, G. R., DNA 5:271-79 (1986) and Kotewicz, M. L., et al., Gene 35:249-58 (1985). M-MLV reverse transcriptase substantially lacking in RNase H activity has also been described. See, e.g., U.S. Pat. No. 5,244,797.

One of the most widely used techniques to study gene expression exploits first-strand cDNA for mRNA sequence(s) as template for amplification by the polymerase chain reaction, PCR. This method, often referred to as RNA PCR or reverse transcriptase PCR (RT-PCR), exploits the high sensitivity and specificity of the PCR process and is widely used for detection and quantification of RNA. Recently, the ability to measure the kinetics of a PCR reaction by on-line detection in combination with these RT-PCR techniques has enabled accurate and precise measurement of RNA sequences with high sensitivity. This has become possible by detecting the RT-PCR product through fluorescence monitoring and measurement of PCR product during the amplification process by fluorescent dual-labeled hybridization probe technologies, such as the “TaqMan” 5′ fluorogenic nuclease assay described by Holland, et al. (Proc. Natl. Acad. Sci. U.S.A. 88, 7276 (1991)), and Gibson, et al. (Genome Res. 6, 99 (1996) or “Molecular Beacons” (Tyagi, S. and Kramer, F. R. Nature Biotechnology 14, 303 (1996)) has described use of dual-labeled hairpin primers. One of the more widely used methods is the addition of double-strand DNA-specific fluorescent dyes to the reaction such as SYBR Green I (Wittwer, et al., Biotechniques 22,130 (1997). These improvements in the PCR method have enabled simultaneous amplification and homogeneous detection of the amplified nucleic acid without purification of PCR product or separation by gel electrophoresis. This combined approach decreases sample handling, saves time, and greatly reduces the risk of product contamination for subsequent reactions, as there is no need to remove the samples from their closed containers for further analysis. The concept of combining amplification with product analysis has become known as “real time” PCR, also referred to as quantitative PCR, or qPCR. The general principles for template quantification by real-time PCR were first disclosed by Higuchi R, G Dollinger, P S Walsh and R. Griffith. Use of real time PCR methods provides a significant improvement towards this goal. However, real-time PCR quantification of mRNA is still bounded by limitations of the process of reverse transcription.

The RT-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with RT-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” RT-PCR procedure (e.g., two step RT-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” RT PCR methods use a common or compromised buffer for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase. Annealing and cDNA synthesis are performed in the presence of Mn⁺⁺ then PCR is carried out in the presence of Mg⁺⁺ after the removal of Mn⁺⁺ by a chelating agent. Finally, the “continuous” method (e.g., one step RT-PCR) integrates the three RT-PCR steps into a single continuous reaction that avoids the opening of the reaction tube for component or enzyme addition.

One step RT-PCR provides several advantages over uncoupled RT-PCR. One step RT-PCR requires less handling of the reaction mixture reagents and nucleic acid products than uncoupled RT-PCR (e.g., opening of the reaction tube for component or enzyme addition in between the two reaction steps), and is therefore less labor intensive, reducing the required number of person hours. One step RT-PCR also requires smaller sample, and reduces the risk of contamination (Sellner and Turbett, 1998). The sensitivity and specificity of one-step RT-PCR has proven well suited for studying expression levels of one to several genes in a given sample or the detection of pathogen RNA. Typically, this procedure has been limited to use of gene-specific primers to initiate cDNA synthesis.

In contrast, use of non-specific primer in the “uncoupled” RT-PCR procedure provides opportunity to capture all RNA sequences in a sample into first-strand cDNA, thus enabling the profiling and quantitative measurement of many different sequences in a sample, each by a separate PCR. The ability to increase the total amount of cDNA produced, and more particularly to produce cDNA that truly represents the mRNA population of the sample would provide a significant advance in study of gene expression. Specifically, such advances would greatly improve the probability of identifying genes which are responsible for disease in various tissues.

Ideally, synthesis of a cDNA molecule initiates at or near the 3′-termini of the mRNA molecules and terminates at the mRNA 5′-end, thereby generating “full-length” cDNA. Priming of cDNA synthesis at the 3′-termini at the poly A tail using an oligo dT primer ensures that the 3′-message of the mRNAs will be represented in the cDNA molecules produced. It would be very desirable if cDNA synthesis initiated at 3′ end and continued to the 5′-end of mRNA's regardless of length of mRNA and the reverse transcriptase used. However, due to many factors such as length, nucleotide sequence composition, secondary structure of mRNA and also inadequate processivity of reverse transcriptases, cDNA synthesis prematurely terminates resulting in non-quantitative representation of different regions of mRNA (i.e., 3′-end sequences or 5′-end sequences). It has been demonstrated that use of mutant reverse transcriptases lacking RNase H activity result in longer cDNA synthesis and better representation, and higher sensitivity of detection. However, it is generally believed that using oligo dT primer results in cDNA sequence bias of mRNA 3′-end region.

In studies involving quantitative analysis of gene expression, sequence bias in the cDNA and non-quantitative representation of different parts of mRNA can yield inaccurate expression data. Due to these problems an alternative method of priming for cDNA synthesis has been used utilizing random primers. Due to random sequence, these primers are believed to non-specifically prime cDNA synthesis at arbitrary sites along the mRNA resulting shorter cDNA fragments that collectively represent all parts of mRNA in the cDNA population. Gerard and D'Alessio (1993 Methods in Molecular Biology 16:73-93) have reported that the ratio of random primer to mRNA is critical for efficient cDNA synthesis by M-MLV RT or its RNase H deficient derivatives. Increasing concentrations of random hexamer resulted in increased yields of cDNA, however, the average length of cDNA decreased accordingly. This indicates that primer concentration must be optimized for different amounts of starting RNA template to achieve efficient cDNA synthesis efficiency. Since random primer has the potential to omit sequence close to the mRNA poly-A tail, in some protocols, oligo dT primer and random primers have been used as mixtures and combine both priming methods.

The choice and concentration of primer can have a profound impact on the quantitative representation of different mRNA transcripts in first-strand cDNA. It is apparent, therefore, that improved compositions and methods for improving the yield of cDNA produced using reverse transcription are greatly to be desired. It is also apparent that new methods for making collections or libraries of cDNA from cells or tissue that more accurately represent the relative amounts of mRNAs present in the cells or tissue are greatly to be desired. It is also apparent that more convenient compositions and kits for use in such methods are desirable.

Accordingly, a need for compositions for synthesizing a cDNA molecule or molecules from an mRNA template or population of mRNA templates under conditions sufficient to increase the detection sensitivity and cDNA yield has been present for a long time. This invention is directed to solve these problems and satisfy a long-felt need.

### SUMMARY OF THE INVENTION

The present invention contrives to solve the disadvantages of the prior art. The present invention provides methods and compositions having trehalose in a concentration between about 5% and about 35% for preparing cDNAs and synthesizing a cDNA molecule or molecules from an mRNA template or population of mRNA templates under conditions sufficient to increase the detection sensitivity and cDNA yield and to simplify and improve the reliability of reverse transcription. Specifically, the invention relates to the use of a mixture of oligo(dT) primer and random primer in a first-strand cDNA synthesis reaction.

The present invention relates to methods or kits for making cDNA molecules, for amplification of RNA by PCR. Compositions according to the present invention comprises mixtures of reagents, including reverse transcriptases, buffers, cofactors and other components, suitable for immediate use in conversion of RNA into cDNA and RT-PCR without dilution or addition of further components. These compositions are useful, alone or in the form of kits, for cDNA synthesis or nucleic acid amplification (e.g., PCR) or for any procedure utilizing reverse transcriptases in a variety of research, medical, diagnostic, forensic and agricultural applications.

The invention also provides improved methods of synthesizing a cDNA molecule(s) from an mRNA templates under conditions sufficient to increase the detection sensitivity and cDNA yield. Specifically, the invention relates to the use of a mixture of oligo(dT) primer and random primer in a first-strand cDNA synthesis reaction.

In one aspect of the invention, the buffer may comprise a monovalent cation selected from the group consisting of Na and K, a magnesium salt, a reducing agent, nucleoside triphosphates, and at least one non-ionic detergent. The buffer may further comprise at least one primer suitable for priming reverse transcription of a template by the reverse transcriptase. The mixture may also comprise an RNase inhibitor protein. In one embodiment, the buffer comprises a potassium salt, a magnesium salt, nucleoside triphosphates, DTT, at least one primer suitable for priming reverse transcription of a template by the reverse transcriptase, at least one non-ionic detergent, and an RNase inhibitor protein.

In any of these methods and compositions, two or more reverse transcriptases may be used, including any reverse transcriptase as described above.

The advantages of the present invention include that (1) the use of trehalose in a concentration between about 5% and about 35% has increased stability of the RT enzyme that is present in the mixture; (2) the compositions of the present invention has increased the detection sensitivity and cDNA yield and simplified and improved the reliability of reverse transcription; (3) in the present invention, some or all of the components of the cDNA synthesis reaction can be combined and stored as a convenient ready-to-use mix that is stable to prolonged storage at −20° C. and that can simply be added to a nucleic acid template solution when needed; and (4) the present invention has provided a more efficient and uniform priming for cDNA synthesis, resulting in more efficient and representative conversion of mRNA sequences into cDNA regardless of distance from 3′ end of mRNA.

Although the present invention is briefly summarized, the fuller understanding of the invention can be obtained by the following drawings, detailed description and appended claims. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows stability information for various reaction mixtures, including mastermixes according to the present invention, for use in reverse transcription reactions;

FIG. 2 shows the results and the efficacy of cDNA synthesis with the mastermixes according to the present invention compared to the reagents stored separately under the conditions recommended in the literature; and

FIG. 3 shows cDNA synthesis with 2×cDNA synthesis mastermixes according to the present invention.

### DETAILED DESCRIPTION OF THE INVENTION

The present invention relates to methods of increasing the efficiency of cDNA synthesis and more particularly, to increasing the sensitivity and accuracy of quantification of gene expression. Compositions of the invention relate to stabilized concentrated reaction mixtures for first-strand cDNA synthesis that simplify and improve the reliability of reverse transcription. Thus, the present invention provides improved cDNA synthesis useful in gene discovery, genomic research, diagnostics and identification of differentially expressed genes and identification of genes of importance to disease.

Use of Primer Combinations

The present invention provides a more efficient and uniform priming for cDNA synthesis. The use of optimal concentration and combinations of random primers and oligo dT provides an efficient and representative conversion of mRNA sequences into cDNA regardless of distance from 3′ end of mRNA. The length of oligo dT can vary from 6 bases to 25 bases and the random primers used according to the present invention (e.g., hexameric, heptameric, octameric, etc.) can vary in size from 6 bases to 9 bases. The amount of random primers can vary from 10 ng to 200 ng for each reaction (20 uL) and that of oligo dT can vary from 2 nM to 50 nM.

Convenient and Stable Reagent Compositions

Another embodiment of the present invention is the form in which the reaction mixture is prepared and stably maintained. Traditionally, cDNA reaction components have been supplied as a number of separate components that are assembled into a complete reaction mix just prior to start of cDNA synthesis primarily due to storage stability issue. A typical kit for cDNA synthesis contains the following components:

Oligo(dT) 12-18 (2.5 uM),
Random hexamers (50 ng),

### RT Buffer (20 mM TrisCl pH 8.4, 50 mM KCl. d. 2.5 mM MgCl₂),

### 10 mM DTT,

0.5 mM dNTPs,
50 units MMLV RT,
40 units RNase inhibitor, and
stabilizer.

Each of the above components is provided separately and frozen at −20° C. for storage. The general belief has been that the components cannot be mixed for long term storage. A key component of these systems is reverse transcriptase that is always stored in special storage buffer with at least 50% glycerol, and is only added to the reaction mix immediately prior to start of cDNA synthesis.

Surprisingly, we have found that some or all of the components of the cDNA synthesis reaction can be combined and stored as a convenient ready-to-use mix that is stable to prolonged storage at −20° C. and that can simply be added to a nucleic acid template solution when needed. The ready to use reaction mixture may contain between about 5% and 35% trehalose to maintain stability of the RT enzyme that is present in the mix.

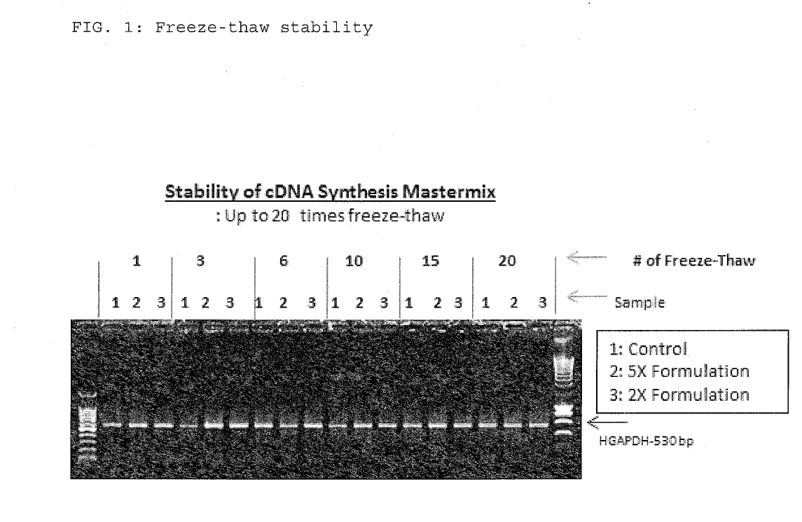

Conventionally, glycerol has been used as an enzyme stabilizer and employed for storage of enzymes. While glycerol based cDNA mixture is not freezing at about −20° C., trehalose based formulation is freezing at about −20° C. and has not been considered as an alternative for storage of enzymes due to the concern on stability issue. However, it has been found in the present invention that trehalose based cDNA can be highly stable under repeated freeze-thaw cycle as shown in FIG. 1.

Various format of cDNA synthesis mastermix can be successfully formulated (1× format to 10× format) for a variety of applications. The minimum components that may usefully be provided for the mixture are the trehalose, the RT and a suitable buffer component. Suitable buffer compounds, such as Tris-HCl, HEPES, etc, are well known in the art. Metal ions necessary for RT activity, such as Mg and a monovalent cation such as K, Na may be present in concentrations that are suitable for RT activity upon addition to a template solution. Additional components that may be present are a reducing agent, such as DTT, primer molecules such as gene specific primers, random primers of any suitable length, oligo(dT) compounds of any suitable length, anchored oligo(dT) molecules of suitable length, detergents or mixtures of detergents such as Tween, NP-40 and equivalent reagents, dNTPs, and one or more RNAse inhibitor proteins. The relative amounts contained in the mixture of such reagents necessary for use in RT reactions, when present, can be readily determined by the skilled artisan. In addition, at least one thermostable DNA polymerase may also be present, which may be used for subsequent PCR reactions or the like.

More specifically, the reagent mixture according to the present invention comprises a ready to use reagent solution that demonstrates prolonged stability when stored at −20° C., wherein the solution comprises (a) trehalose in a concentration between about 5% and about 35%, and (b) a viral reverse transcriptase in a concentration sufficient for use in a reverse transcription reaction without adding additional reverse transcriptase, wherein the viral reverse transcriptase is selected from the group consisting of AMV RT, RSV RT, MMLV RT, HIV RT, EIAV RT, RAV2 RT, ASLV RT, and RNaseH (−) RT such as SuperScript II RT and ThermoScript RT, in a buffer suitable for use in a reverse transcription reaction, wherein the buffer comprises a co-factor metal ion necessary for reverse transcriptase activity and nucleoside triphosphates. Preferably, the concentration of trehalose may be between about 10% and about 35% for better stability of the mixture.

The buffer may further comprise at least one primer suitable for priming reverse transcription of a template by the reverse transcriptase. The buffer may include an RNase inhibitor protein as well. Moreover, the buffer may additionally include a potassium salt, a magnesium salt, nucleoside triphosphates, DTT, at least one primer suitable for priming reverse transcription of a template by said reverse transcriptase, at least one non-ionic detergent, and an RNase inhibitor protein. The solution of the present invention may further comprise an additional viral reverse transcriptase enzyme(s).

Besides, the buffer may further comprise at least one random primer (e.g., hexameric, heptameric, octameric, etc.) and/or at least one oligo dT primer (e.g., dT-6-10, dT-12-18, etc.). The metal ion necessary for reverse transcriptase activity may be magnesium ion and the buffer may comprise a monovalent cation (e.g., K, Na, etc.). The buffer may further comprise a reducing agent (e.g., DTT, etc.) and/or a non-ionic detergent (e.g., NP-40, Tween-20, etc.). Furthermore, the solution of the present invention is stable for 1 month to 2 years when stored at −20° C.

Accordingly, the present invention provides newly improved, convenient, and ready to use configurations for cDNA synthesis. The methods of the invention reduce the number of additions for assembly of cDNA synthesis reactions which is highly sought by researchers especially in high throughput applications.

According to the methods of the invention, the ready to use mixes for cDNA synthesis can be made at different concentrations and provided as 1× to 10×“mastermixes.” The following is an example of a 2× mastermix for cDNA synthesis that contains all components necessary for cDNA synthesis according to the methods of this invention. Using 10 uL of this mastermix and RNA preparation of interest at a total volume of 20 uL provides a complete reaction mix for conversion of RNA into cDNA.

Formulation for 2×cDNA Synthesis Mastermix:

### 2× Buffer (40 mM Tris-HCl, pH 8.4, 0.1M KCl),

10 mM MgCl₂ 1 mM dNTP (each) 20 mM DTT,
1 nM oligo(dT)₂₀ 100 ng random primer 19% Trehalose,

### 0.005% NP-40,

100 units of MMLV RT, and
4 units RNase inhibitor protein.

In addition to the above formulation, three other mastermixes were prepared that contained all reagents except the primers.

cDNA Synthesis Mastermix 1 did not have primers.

cDNA Synthesis Mastermix 2 contained oligo dT as the primers.

cDNA Synthesis Mastermix 3 contained random hexamers and octamers as primers.

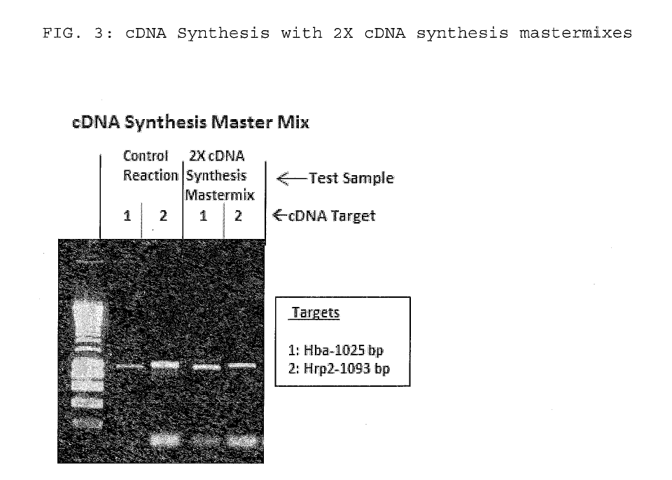

All of the above 2×cDNA synthesis mastermixes were found to be stable for months when stored at −20° C. FIG. 3 shows the results and the efficacy of cDNA synthesis with these mastermixes.

It will be evident to those skilled in the art that a variety of different reverse transcriptases can be used according to the method of the invention. The reverse transcriptases may include, without limitation, AMV RT, RSV RT, MMLV RT, RNase H-mutants of various reverse transcriptases, HIV RT, EIAV RT, RAV2 RT, TTH DNA polymerase, C. hydrogenoformans DNA polymerase, Superscript II RT, SuperScript RT, ThermoScript RT and mixtures thereof. It will also be obvious that one or more of the components of the above mastermix can be substituted with other equivalent reagent or protein. For example, there are a number of different RNase inhibitor proteins that can be used. Thermostable DNA polymerases suitable for use in the mastermixes are well known in the art and include Taq, Tth, Tne, Tma, Tli, Pfu, Pwo, Bst, Bca, Sac, Tac, Tfl, Tru, Mth, Mtb, and Mlep DNA polymerases and the like.

The composition of the 2× buffer provided can also be varied, for example, by use of other buffers such as sulfate containing buffers or acetate based buffers that have been used for cDNA synthesis. It will be apparent to those skilled in the art that different formulations can be optimized for different applications.

### EXAMPLES

### FIG. 1: Freeze-Thaw Stability

Test samples were: (1) Control cDNA synthesis Kit (Legene), (2) cDNA Synthesis Mastermix (5× formulation), and (3) SM-B: cDNA Synthesis Mastermix (2× formulation). Samples were subjected to number of freeze-thaw cycles as indicated (used dry-ice and room temperature thaw) and placed all samples on ice. Reactions were assembled by adding 1 ng, 10 ng of total HeLa RNA as indicated. RT reaction was carried out at 40° C. 30 minutes incubation, heat-killed RT upon incubation at 85° C. for 5 minutes and placed on ice. PCR reactions were assembled with 2×PCR Mix (LeGene). Added 2 ul of RT sample each and GAPDH-530 bp primers and followed by 36 cycles of PCR amplification (94° C., 15 s, 60° C., 30 s, 72° C., 1 min) and analyzed the amplified products by 1% agarose gel electrophoresis.

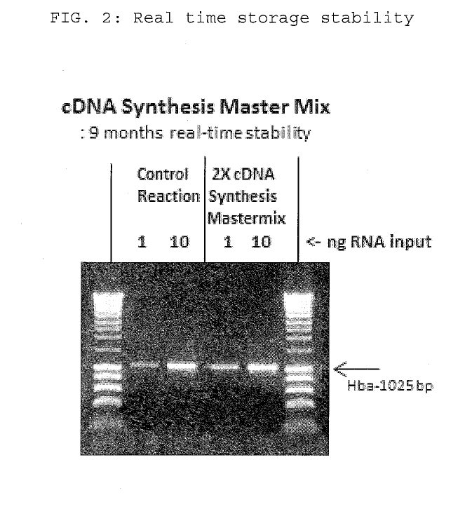

### FIG. 2: Real Time Storage Stability

9 months old samples were removed from −20° C. freezer. Test samples were: (1) Control cDNA synthesis Kit (Legene), and (2) cDNA Synthesis Mastermix (2× formulation). Reactions were assembled by adding 1 ng, 10 ng of total HeLa RNA as indicated. RT reaction was carried out at 40° C. 30 minutes incubation, heat-killed RT upon incubation at 85° C. for 5 minutes and placed on ice. PCR reactions were assembled with 2×PCR Mix (Legene). Added 2 ul of RT sample each and Hba-1025 bp primers and followed by 36 cycles of PCR amplification (94° C., 15 s, 60° C., 30 s, 72° C., 1 min) and analyzed the amplified products by 1% agarose gel electrophoresis.

### FIG. 3: cDNA Synthesis with 2×cDNA Synthesis Mastermixes

Test samples were: (1) Control cDNA synthesis Kit (Legene), and (2) cDNA Synthesis Mastermix (2× formulation). Reactions were assembled by adding 1 ng (for Hba-1025 bp test), 10 ng (for Hrp2-1093 test) of total HeLa RNA as indicated. RT reaction was carried out at 40° C. 30 minutes incubation, heat-killed RT upon incubation at 85° C. for 5 minutes and placed on ice. PCR reactions were assembled with 2×PCR Mix (LeGene). Added 2 ul of RT sample each and Hba-1025 bp primers and Hrp2-1093 bp primer as indicated then followed by 36 cycles of PCR amplification (94° C., 15 s, 60° C., 30 s, 72° C., 1 min) and analyzed the amplified products by 1% agarose gel electrophoresis.

According to the tests, the 2× and 5×cDNA synthesis mastermixes were found to be stable for more than 9 months when stored at −20° C.

While the invention has been shown and described with reference to different embodiments thereof, it will be appreciated by those skilled in the art that variations in form, detail, compositions and operation may be made without departing from the spirit and scope of the invention as defined by the accompanying claims.

What is claimed is:

1. A reagent mixture comprising a ready to use reagent solution, wherein the solution comprises:
(a) trehalose in a concentration between about 5% and about 35%; and
(b) a viral reverse transcriptase in a concentration sufficient for use in a reverse transcription reaction without adding additional reverse transcriptase, wherein the viral reverse transcriptase is selected from the group consisting of AMV RT, RSV RT, MMLV RT, HIV RT, EIAV RT, RAV2 RT, ASLV RT, RNaseH (−) RT, SuperScript II RT, and ThermoScript RT,
in a buffer suitable for use in a reverse transcription reaction, wherein the buffer further comprises:
a co-factor metal ion necessary for reverse transcriptase activity; and
nucleoside triphosphates.

2. The mixture according to claim 1, wherein the buffer further comprises at least one primer suitable for priming reverse transcription of a template by the reverse transcriptase.

3. The mixture according to claim 1, wherein the buffer comprises an RNase inhibitor protein.

4. The mixture according to claim 1, wherein the buffer comprises a potassium salt, a magnesium salt, nucleoside triphosphates, DTT, at least one primer suitable for priming reverse transcription of a template by the reverse transcriptase, at least one non-ionic detergent, and an RNase inhibitor protein.

5. The mixture according to claim 1, wherein the solution comprises at least two viral reverse transcriptase enzymes.

6. The mixture according to claim 1, wherein the buffer comprises at least one random primer.

7. The mixture according to claim 1, wherein the buffer comprises hexameric, heptameric, or octameric.

8. The mixture according to claim 1, wherein the buffer further comprises oligo dT primer.

9. The mixture according to claim 1, wherein the buffer further comprises dT-6-10 or dT-12-18.

10. The mixture according to claim 1, wherein the buffer further comprises at least one random primer and at least one oligo dT primer.

11. The mixture according to claim 1, wherein the metal ion necessary for reverse transcriptase activity is magnesium ion.

12. The mixture according to claim 1, wherein the buffer comprises a monovalent cation.

13. The mixture according to claim 1, wherein the buffer comprises K or Na.

14. The mixture according to claim 1, wherein the buffer comprises a reducing agent.

15. The mixture according to claim 1, wherein the buffer comprises DTT.

16. The mixture according to claim 1, wherein the buffer comprises a non-ionic detergent.

17. The mixture according to claim 1, wherein the buffer comprises NP-40 or Tween-20.

18. The mixture according to claim 1, wherein the solution is stable for 1 month to 2 years when stored at −20° C.

19. The mixture according to claim 1, wherein the trehalose is in a concentration between about 10% and about 35%.
